(19) United States
(12) Patent Application Publication
(10) Pub. No.: US 2023/0351343 A1
(43) Pub. Date: Nov. 2, 2023

(54) DISTRIBUTED RULES BASED HIERARCHICAL AUDITING SYSTEM FOR MANAGEMENT OF HEALTH CARE SERVICES

(71) Applicant: Magnum Transaction Sub, LLC, Nashville, TN (US)

(72) Inventors: TRACY WEBER, Longboat Key, FL (US); CHRISTINE BELANGER, Wilton, CT (US); DANIEL FITZGERALD, Lansdale, PA (US); MARIAN COONEY, Glenmoore, PA (US); SERGEY POLVAKOV, Norwalk, CT (US); MARK KESSLER, Brookhaven, PA (US); KRISHNA KUSUMBA, West Chester, PA (US); NIRMALKUMAR PALANISAMY, Karnataka (IN)

(73) Assignee: Magnum Transaction Sub, LLC, Nashville, TN (US)

(21) Appl. No.: 18/348,540

(22) Filed: Jul. 7, 2023

Related U.S. Application Data
(63) Continuation of application No. 17/038,640, filed on Sep. 30, 2020.

(51) Int. Cl. G06Q 20/08; G16H 40/63; G16H 40/67; G16H 20/00

(52) U.S. Cl. CPC G06Q 20/0855; G16H 40/63; G16H 40/67; G16H 20/00

(57) ABSTRACT

Receiving information associated with a health care service provided to a patient; distributing a plurality of rules for determining responsibility for payment for the health care service across a plurality of hierarchical computer-implemented auditing systems including a primary auditing system and a secondary auditing system; generating, using the primary auditing system, a first payment recommendation for the health care service based on a first subset of the plurality of rules and the information associated with the health care service; communicating the information associated with the health care service to the secondary auditing system; generating, using the secondary auditing system, a second payment recommendation for the health care service based on a second subset of the plurality of rules and the information associated with the health care service; and merging the first payment recommendation with the second payment recommendation to generate a final payment recommendation for the health care service.

### FIELD

The present inventive concepts relate generally to health care systems and services and, more particularly, management of claims for health care services processed by payor entities.

### BACKGROUND

Health care services are delivered to patients through health care providers, e.g., one or more medical practitioners. Payment for these services is usually made by one or more payors, which may include the patient and another entity, such as an insurance company. Both private companies and public organizations, such as the Medicare and Medicaid programs run by the federal government, and public employee programs run by the federal government or states, may act as payors. Payors may be penalized for improperly denying payment for health care service claims, but may also be harmed financially for approving payment for claims that cannot be justified. To improve the accuracy in determining which claims to pay, which claims to pay in part, and which claims to deny, payors may use an auditing system that provides recommendations on the payment process. The auditing system may be designed with various packages of rules that can be applied to the claims to make the recommendations on payment. A payor, however, may not have licensed all of the various rules because they are not frequently used or because of the costs associated with licensing certain rules packages. In addition, a payor may upgrade the auditing system rules on a fixed schedule, which may be infrequent. This may delay a payor gaining access to new rules that have been developed based on regulatory standards and best practices.

### SUMMARY

According to some embodiments of the inventive concept, a method comprises receiving information associated with a health care service provided to a patient; distributing a plurality of rules for determining responsibility for payment for the health care service across a plurality of hierarchical auditing systems including a primary auditing system and a secondary auditing system; generating, using the primary auditing system, a first payment recommendation for the health care service based on a first subset of the plurality of rules and the information associated with the health care service; communicating the information associated with the health care service to the secondary auditing system; generating, using the secondary auditing system, a second payment recommendation for the health care service based on a second subset of the plurality of rules and the information associated with the health care service; and merging the first payment recommendation with the second payment recommendation to generate a final payment recommendation for the health care service.

In other embodiments, the first payment recommendation is a recommendation not to pay for the health care service. Merging the first payment recommendation with the second payment recommendation to generate the final payment recommendation for the health care service comprises generating the final payment recommendation as the first payment recommendation.

In still other embodiments, the method further comprises associating a tag with the information associated with the health care service. The tag provides instructions for processing information associated with the health care service in the secondary auditing system.

In still other embodiments, the information is first information, the health care service is a first health care service, and the tag is a first tag. The method further comprises receiving second information associated with a second health care service provided to the patient; associating a second tag with the second information associated with the second health care service, the second tag providing instructions for processing the second information associated with the second health care service in the secondary auditing system; generating, using the primary auditing system, a first payment recommendation for the second health care service based on the first subset of the plurality of rules and the second information associated with the second health care service; generating, using the secondary auditing system, a second payment recommendation for the second health care service based on the second subset of the plurality of rules, the second information associated with the second health care service, the second tag, the first information associated with the first health care service, and the first tag; and merging the first payment recommendation for the second health care service with the second payment recommendation for the second health care service to generate a final payment recommendation for the second health care service.

In still other embodiments, the first payment recommendation is a recommendation not to pay for the health care service.

In still other embodiments, the information associated with the health care service corresponds to a line item in a health care claim for payment; and communicating the information associated with the health care service to the secondary auditing system comprises communicating the health care claim for payment to the secondary auditing system.

In still other embodiments, the information associated with the health care service corresponds to a line item in a health care claim for payment; and communicating the information associated with the health care service to the secondary auditing system comprises communicating the line item to the secondary auditing system without communicating other line items of the health care claim for payment to the secondary auditing system

In still other embodiments, the first payment recommendation is a recommendation to pay for the health care service in whole or in part. The second payment recommendation is not to pay in whole or in part for the health care service. Merging the first payment recommendation with the second payment recommendation to generate the final payment recommendation for the health care service comprises generating the final payment recommendation as the second payment recommendation when the second payment recommendation is an amount less than the first payment recommendation.

In still other embodiments, the first payment recommendation is a recommendation to pay for the health care service in whole or in part based on the first subset of the plurality of rules, the information associated with the health care service and historical information associated with the health care service, which is unavailable to the secondary auditing system. The second payment recommendation is not to pay in whole or in part for the health care service. Merging the first payment recommendation with the second payment recommendation to generate the final payment recommendation for the health care service comprises generating the final payment recommendation as the first payment recommendation.

In some embodiments of the inventive concept, a system comprises a processor and a memory coupled to the processor and comprising computer readable program code embodied in the memory that is executable by the processor to perform operations comprising: receiving information associated with a health care service provided to a patient; distributing a plurality of rules for determining responsibility for payment for the health care service across a plurality of hierarchical auditing systems including a primary auditing system and a secondary auditing system; generating, using the primary auditing system, a first payment recommendation for the health care service based on a first subset of the plurality of rules and the information associated with the health care service; communicating the information associated with the health care service to the secondary auditing system; generating, using the secondary auditing system, a second payment recommendation for the health care service based on a second subset of the plurality of rules and the information associated with the health care service; and merging the first payment recommendation with the second payment recommendation to generate a final payment recommendation for the health care service.

In further embodiments, the first payment recommendation is a recommendation not to pay for the health care service. Merging the first payment recommendation with the second payment recommendation to generate the final payment recommendation for the health care service comprises generating the final payment recommendation as the first payment recommendation.

In still further embodiments, the operations further comprise associating a tag with the information associated with the health care service, the tag providing instructions for processing the information associated with the health care service in the secondary auditing system.

In still further embodiments, the information is first information, the health care service is a first health care service, and the tag is a first tag, the operations further comprising: receiving second information associated with a second health care service provided to the patient; associating a second tag with the second information associated with the second health care service, the second tag providing instructions for processing the second information associated with the second health care service in the secondary auditing system; generating, using the primary auditing system, a first payment recommendation for the second health care service based on the first subset of the plurality of rules and the second information associated with the second health care service; generating, using the secondary auditing system, a second payment recommendation for the second health care service based on the second subset of the plurality of rules, the second information associated with the second health care service, the second tag, the first information associated with the first health care service, and the first tag; and merging the first payment recommendation for the second health care service with the second payment recommendation for the second health care service to generate a final payment recommendation for the second health care service.

In still further embodiments, the first payment recommendation is a recommendation not to pay for the health care service.

In still further embodiments, the information associated with the health care service corresponds to a line item in a health care claim for payment; and communicating the information associated with the health care service to the secondary auditing system comprises communicating the health care claim for payment to the secondary auditing system.

In some embodiments of the inventive concept, a computer program product, comprises a non-transitory computer readable storage medium comprising computer readable program code embodied in the medium that is executable by a processor to perform operations comprising: receiving information associated with a health care service provided to a patient; distributing a plurality of rules for determining responsibility for payment for the health care service across a plurality of hierarchical auditing systems including a primary auditing system and a secondary auditing system; generating, using the primary auditing system, a first payment recommendation for the health care service based on a first subset of the plurality of rules and the information associated with the health care service; communicating the information associated with the health care service to the secondary auditing system; generating, using the secondary auditing system, a second payment recommendation for the health care service based on a second subset of the plurality of rules and the information associated with the health care service; and merging the first payment recommendation with the second payment recommendation to generate a final payment recommendation for the health care service.

In other embodiments, the first payment recommendation is a recommendation not to pay for the health care service. Merging the first payment recommendation with the second payment recommendation to generate the final payment recommendation for the health care service comprises generating the final payment recommendation as the first payment recommendation.

In still other embodiments, the operations further associating a tag with the information associated with the health care service, the tag providing instructions for processing the information associated with the health care service in the secondary auditing system.

In still other embodiments, the information is first information, the health care service is a first health care service, and the tag is a first tag, the operations further comprising: receiving second information associated with a second health care service provided to the patient; associating a second tag with the second information associated with the second health care service, the second tag providing instructions for processing the second information associated with the second health care service in the second auditing system; generating, using the primary auditing system, a first payment recommendation for the second health care service based on the first subset of the plurality of rules and the second information associated with the second health care service; generating, using the secondary auditing system, a second payment recommendation for the second health care service based on the second subset of the plurality of rules, the second information associated with the second health care service, the second gat, the first information associated with the first health care service, and the first tag; and merging the first payment recommendation for the second health care service with the second payment recommendation for the second health care service to generate a final payment recommendation for the second health care service.

In still other embodiments, the first payment recommendation is a recommendation not to pay for the health care service.

It is noted that aspects described with respect to one embodiment may be incorporated in different embodiments although not specifically described relative thereto. That is, all embodiments and/or features of any embodiments can be combined in any way and/or combination. Moreover, other methods, systems, articles of manufacture, and/or computer program products according to embodiments of the inventive concept will be or become apparent to one with skill in the art upon review of the following drawings and detailed description. It is intended that all such additional systems, methods, articles of manufacture, and/or computer program products be included within this description, be within the scope of the present inventive subject matter, and be protected by the accompanying claims. It is further intended that all embodiments disclosed herein can be implemented separately or combined in any way and/or combination.

### BRIEF DESCRIPTION OF THE DRAWINGS

Other features of embodiments will be more readily understood from the following detailed description of specific embodiments thereof when read in conjunction with the accompanying drawings, in which:

FIG. 1 is a block diagram of a hierarchical auditing system for health care service claims in accordance with some embodiments of the inventive concept;

FIG. 2 is a block diagram that illustrates a communication network including a hierarchical auditing system, which includes a primary auditing system, a secondary auditing system, and a remote connection system, for generating processing recommendations for health care service claims in accordance with some embodiments of the inventive concept;

FIGS. 3-9 are flowcharts that illustrates operations for generating processing recommendations for health care service claims using the primary auditing system and secondary auditing system of FIG. 2 in accordance with some embodiments of the inventive concept;

FIG. 10 is a data processing system that may be used to implement one or more servers in the hierarchical auditing system of FIGS. 1 and 2 in accordance with some embodiments of the inventive concept;

FIG. 11 is a block diagram that illustrates a software/hardware architecture for use in the primary auditing system of FIGS. 1 and 2 in accordance with some embodiments of the inventive concept;

FIG. 12 is a block diagram that illustrates a software/hardware architecture for use in the secondary auditing system of FIGS. 1 and 2 in accordance with some embodiments of the inventive concept; and

FIG. 13 is a block diagram that illustrates a software/hardware architecture for use in the remote connection system of FIGS. 1 and 2 in accordance with some embodiments of the inventive concept.

### DETAILED DESCRIPTION

In the following detailed description, numerous specific details are set forth to provide a thorough understanding of embodiments of the present inventive concept. However, it will be understood by those skilled in the art that the present invention may be practiced without these specific details. In some instances, well-known methods, procedures, components and circuits have not been described in detail so as not to obscure the present inventive concept. It is intended that all embodiments disclosed herein can be implemented separately or combined in any way and/or combination. Aspects described with respect to one embodiment may be incorporated in different embodiments although not specifically described relative thereto. That is, all embodiments and/or features of any embodiments can be combined in any way and/or combination.

As used herein, the term “provider” may mean any person or entity involved in providing health care services to a patient.

Some embodiments of the inventive concept are described with reference to a payor determining whether to make payment for a claim for services or products. In the context of a health care environment, a payor may be, for example, an entity that provides health or medical insurance, such as private insurance companies and government insurance agencies, both at the federal and state levels (e.g., Medicare, Medicaid, public employee insurance agencies, and the like). Health care providers may submit claims for medical services rendered, products prescribed (e.g., medications, medical devices, etc.), administrative fees, and/or other fees or expenses to a payor for payment. Upon receiving a claim, the payor may then determine whether to pay the claim in whole, in part, or deny the claim. A claim may include both header and line information. The header may be information that applies to the entire claim, e.g., patient details (name, date of birth, address, etc.). The line information may identify the various services, products, fees, expenses, and/or other items for which payment is sought and may be referred to as line item(s). Thus, a payor may evaluate each line in the claim to determine whether to pay the invoiced amount in full, in part, or to deny payment for that line.

A payor, such as an insurance company operating in the health care field, may make use of a claims auditing system to assist them in determining whether to pay and how much to pay for the various lines listed in claims submitted for payment. As described above, payors may be penalized for improperly denying payment of a claim. But a payor may suffer economically if payment is made for fraudulent claims or claims for which reimbursement has not been authorized or contracted for. Some embodiments of the inventive concept stem from a realization that the claims auditing system used by a payor may only contain a subset of all the rules that may be available for evaluating claims for payment. For example, the rules may be packaged together in groups, which are sometimes referred to as knowledge packs, and a payor may only license or purchase a portion of the rules. In some circumstances, the payor may wish to have access to more rules, but schedules updates to the claim auditing system on a fixed schedule, which may result in an extended time period before new rules can be introduced into the system. According to some embodiments of the inventive concept, a secondary auditing system may be provided that includes additional rules for evaluating claims for payment that are not available to the payor by way of the primary auditing system. The secondary auditing system may be provided, for example, remotely through a cloud service. A remote connection system may provide an interface between the primary auditing system and the secondary auditing system. The remote connection system may act as a filter at the claim level for determining whether to forward a claim including the line items therein to a secondary auditing system. The remote connection system may not forward a claim to the secondary auditing system based on one or more claim level fields that may be indicative of whether a secondary auditing system may provide value. For example, a claim level field may be defined that provides an identification for the claims processing system used by a payor. If the claims processing system is not configured to accept a recommendation from a secondary auditing system, then the remote connection system need not forward the claim to the secondary auditing system. Similarly, a field may be defined indicating whether a patient is self-insured or non-self-insured. The remote connection system may not forward claims associated with patients who are self-insured. In some embodiments, when the primary auditing system makes a determination to assign a do not pay status to one or more line items in a claim, then the secondary auditing system will not be tasked with making a further recommendation. Nevertheless, line items in a claim that have been assigned a do not pay status by the primary auditing system may be useful to the secondary auditing system in evaluating line items in the same claim or other claims for payment.

Thus, some embodiments of the inventive concept provide a tagging mechanism in which a tag can be associated with a line item in a claim. The tag can assume multiple states and can be used to provide more nuance to the general rule that denied line items are not forwarded to the secondary auditing system for evaluation. A tag may be viewed as an instruction or a mechanism for conveying information that may be used by a rules engine running on the secondary auditing system for evaluating the line item with the tag or for using the tagged line item to evaluate another line item in the same claim or another claim. For example, if the primary auditing system recommends denying payment for a line item of a claim with high certainty, then this line item may be useful to the secondary auditing system in evaluating line items in the present claim or other claims even though the secondary auditing system would not be tasked with making a payment recommendation on that tagged line item because of the primary auditing system recommendation of denying payment. Conversely, if the primary system recommends denying payment for a line item of a claim with less certainty, including those instances where the primary auditing system recommends a person perform a manual review of the line item, then the tag state may be set to prohibit or limit the secondary auditing system's use of the line item in evaluating other claims and other line items due to the uncertainty surrounding present line item. Thus, some embodiment of the inventive concept provide for sending claims to a secondary auditing system in whole or not at all based on claim level field information. By sending or not sending entire claims, the primary system need not split apart claims by line with some lines being sent to a secondary auditing system and others filtered and not sent to the secondary auditing system. This reduces the processing power needed to divided the claims into individual lines and re-assemble the claims once a secondary auditing system has completed making recommendations on the lines that were sent to it. Other embodiments, however, may allow for the division of claims into lines with decisions being made at the claim line level of which lines to send to a secondary auditing system for evaluation.

The remote connection system may also be used to merge the recommendations between the primary auditing system and the secondary auditing system including resolving any conflicts that may occur due to the primary auditing system having access to more or different historical claim data than may be available to the secondary auditing system. A primary or secondary auditing system may access claim history from an Operational Data Store (ODS) database and/or from their claims processing system. Moreover, a primary auditing system may pass claim history information on to a secondary auditing system. A payor may control how much or how little claim history is passed to a secondary auditing system. As a result, a primary auditing system and a secondary auditing system may have different history information. In some embodiments, the payor may elect to only communicate historical claim information associated with paid claims because a paid claim is typically a final decision making the most accurate. Although described herein in the context of a primary auditing system and a secondary auditing system for evaluating claims for payment, the system architecture may be viewed as a hierarchical auditing system including a primary auditing system and one or more secondary auditing system. Multiple secondary auditing systems may be connected in series in a daisy-chain manner with a remote connection system supporting the addition of each new secondary auditing system to the chain. Rules may be developed including the use of tags to determine which line items are forwarded to each successive secondary auditing system. According to some embodiments described herein, the rules used for evaluating claims for payment may be distributed over a hierarchy of auditing systems beginning with a primary auditing system and one or more secondary auditing systems. The rules applied to claim line items via a secondary auditing system may result in the denial of payment or a reduction in payment amount for one or more line items that otherwise would have been approved by the primary auditing system thereby providing savings to the payor. The payor may subscribe or license the rules provided in a secondary auditing system on a contingency basis where the payor is charged a percentage of the savings that are obtained through use of the secondary auditing system. Because the secondary auditing system may be provided as a service, such as a cloud service, for example, a payor may gain access to new rules on an expedited basis without having to wait to update the rules in the primary auditing system.

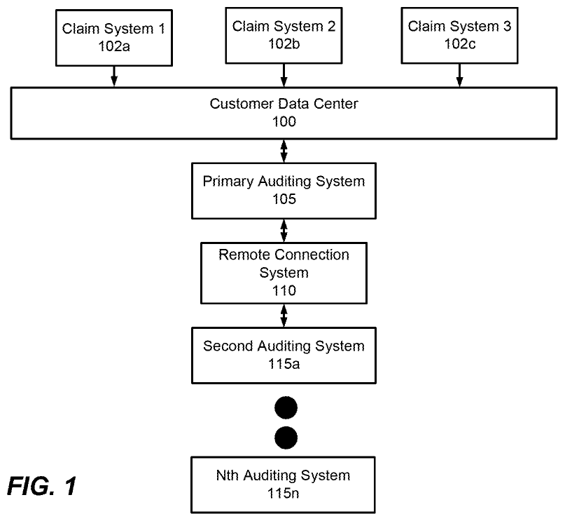

FIG. 1 is a block diagram of a hierarchical auditing system for health care service claims in accordance with some embodiments of the inventive concept. Referring to FIG. 1, a payor, such as an insurance entity operating in the health care field, may have a customer data center 100 through which claims are evaluated and processed for payment. The claims may be received through one or more claims systems 102a, 102b, and 102c, such as claim system 1 102a, claim system 2 102b, and claim system 3, 102c. Each of these claim systems 102a, 102b, and 102c may be used to receive and coordinate payment for claims generated by providers (e.g., health care or medical service providers). The claim systems 102a, 102b, and 102c may use different formats, but the claims may each include header information and line information or line items as described above. The payor may use the customer data center 100 to organize the claims and evaluate whether to pay the claims in whole, in part, or to deny the claims. The customer data center 100 may be representative of an entity for handling or processing claims. In the example embodiments provided herein, the claims systems may be integrated into the primary auditing system 105 and the customer data center 100 may provide a user interface. Each claim system may be independent of the other and have its own primary and secondary auditing system as well as its own user interface. The payor may use a primary auditing system 105, which contains rules for evaluating the claims for payment (in whole or part) or non-payment (claim denial or rejection). The payor may also make use of one or more secondary auditing systems, which are shown as a second auditing system 115a through an Nth auditing system 115n in FIG. 1. These secondary auditing systems 115a, . . . , 115n may be linked serially through one or more remote connection systems 110. The secondary auditing systems 115a, . . . , 115n may be accessed, for example, through the cloud and may provide access to rules for evaluating claims for payment that are not available to the payor by way of the primary auditing system 105. The remote connection system 110 may be configured to serve as a proxy for forwarding claims to a secondary auditing system 115a, . . . , 115n for evaluation for payment. In this regard, the remote connection system 110 may provide a tagging capability in which a line item may have a tag associated therewith that can be configured in one or more states to provide instructions, which can then be processed by rules running on one or more of the secondary auditing systems 115a, . . . , 115n in the hierarchy to evaluate the tagged line for payment or for use in support in evaluating other lines in the same claim and/or other claims. The remote connection system 110 may also be configured to merge the payment recommendation between the primary auditing system 105 and one or more of the secondary auditing systems 115a, . . . , 115n.

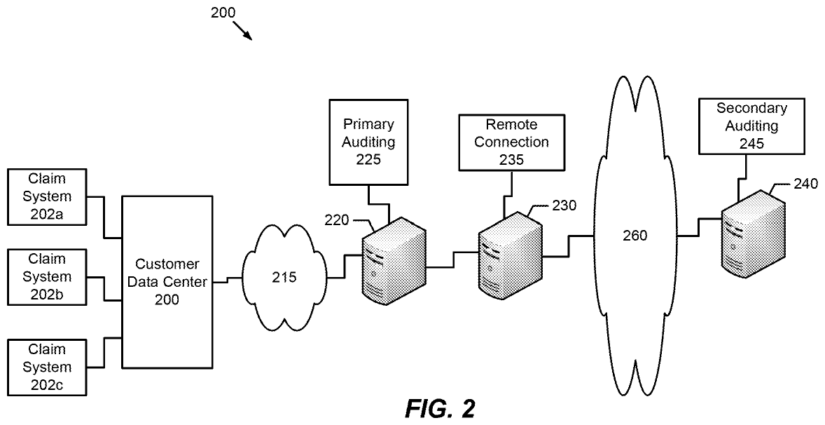

Referring to FIG. 2, a communication network including a hierarchical auditing system, which includes a primary auditing system, a secondary auditing system, and a remote connection system, for generating processing (e.g., payment) recommendations for health care service claims comprises a customer (e.g., payor) data center 200 that is coupled to one or more claim systems 202a, 202b, and 202c and also coupled to a primary auditing system server 220 via a network 215. Each of the claim systems 202a, 202b, and 202c may be used to receive and coordinate payment for claims generated by providers (e.g., health care or medical service providers). The claim systems 202a, 202b, and 202c may use different formats, but the claims may each include header information and line information or line items as described above. The customer data center 200 may be a data center including one or more servers associated with a payor, such as an insurance entity operating in the health care field. The primary auditing system server 220 may be configured to assist the customer data center 200 in evaluating whether to pay, pay in part, or deny payment for line items in claims that are processed for payment in the customer data center 200. The primary auditing server 220 may include a primary auditing module 225 that includes rules for evaluating and making payment recommendations for the line items in the claims. The primary auditing module 225 may also include history information on past claims received and processed, which can be used in evaluating new claims for payment. For example, if a patient is limited to a certain number of physical therapy sessions, then historical claims may be consulted to determine if a patient has reached a limit for which payment is authorized. As described above, a payor may not subscribe or license all of the rules available for evaluating claims for payment. In addition, a payor may schedule updates for rules running on the primary auditing system server 220 infrequently, which may deprive the payor to access of more recent rules that have been developed for evaluating line items in claims for payment.

To allow the payor to access additional rules for evaluating claim line items for payment in an expedited fashion, a remote connection system server 230 may be used to provide access to a secondary auditing system server 240. The secondary auditing system server 240 includes a secondary auditing module 245, which includes additional rules not available via the primary auditing module 225 for evaluating claim line items for payment, payment in part, or non-payment. The secondary auditing module 245 may also include some history information for claims and/or line items that have been previously evaluated and processed. The remote connection system server 230 includes a remote connection module 235 and may be configured to serve as a proxy for filtering and forwarding claims to a secondary auditing system 115a, . . . , 115n for evaluation for payment. In some embodiments, the remote connection system server 230 may provide a tagging capability to provide instructions that may be processed using the rules running on the secondary auditing system server 240. The various states associated with a tag may be interpreted by the rules running on the secondary auditing system 240 to evaluate the present tagged line for payment and/or use the present tagged line to evaluate another line for payment in the same claim and/or different claim. For example, a line item of a claim that has been recommended for non-payment by the primary auditing system server 220 may be processed by secondary auditing system server 240 based on the state of a tag associated therewith. If the certainty associated with the non-payment recommendation is high, then a tag may be associated with the line item and configured in a state that allows the secondary auditing system server 240 to use the information in the line item as support in evaluating one or more other line items in the same claim or other claims for payment. Conversely, if the non-payment recommendation generated by the primary auditing module 225 is made with less certainty, including circumstances where a manual review is recommended, then the tag state may be set to prohibit or limit the secondary auditing module's 245 use of the line item in evaluating other claims and other line items due to the uncertainty associated with the primary auditing module's 225 recommendation. The remote connection system server 240 may also be configured to merge the payment recommendation between the primary auditing system server 220 the secondary auditing system server 240.

The customer data center 200 of FIG. 2 and the customer data center 100 of FIG. 1 may be configured with a user interface that allows a customer, e.g., a payor to view the recommendations generated by the primary auditing system server 220 and the secondary auditing system server 240 for claims and the individual line items contained therein. The primary auditing module 225 may have references to all rules used in the secondary auditing module 245. In general, an auditing system higher in the hierarchy may have references to all rules used by auditing systems used lower in the hierarchy. In some embodiments, the user interface may allow a payor to review which specific rules have been triggered in the primary auditing system server 220 and/or the secondary auditing system server 245 in making a recommendation. This may allow a payor to directly see the benefit of individual rules on each auditing system in the hierarchy in generating potential savings when processing claims for payment.

A network 260 couples the primary auditing system server 220 and remote connection server 230 to the secondary auditing system server 240. The network 260 may be a global network, such as the Internet or other publicly accessible network. Various elements of the network 260 may be interconnected by a wide area network, a local area network, an Intranet, and/or other private network, which may not be accessible by the general public. Thus, the communication network 260 may represent a combination of public and private networks or a virtual private network (VPN). The network 260 may be a wireless network, a wireline network, or may be a combination of both wireless and wireline networks.

Although the primary auditing system server 220, remote connection system server 230, and the secondary auditing system server 240 are shown as separate physical servers, it will be understood that the functionality and capabilities of the various servers may be logically combined/divided among one or more physical servers.

Although FIG. 2 illustrates an example communication network including a hierarchical auditing system for generating recommendations for health care service claims, it will be understood that embodiments of the inventive subject matter are not limited to such configurations, but are intended to encompass any configuration capable of carrying out the operations described herein.

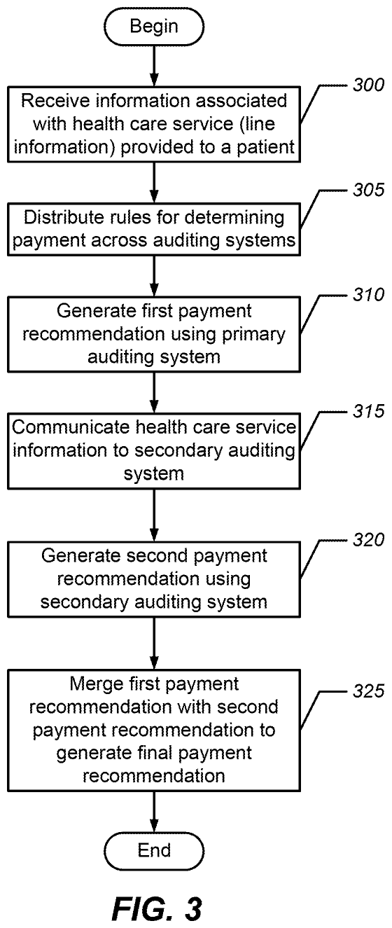

FIGS. 3-9 are flowcharts that that illustrate operations for generating processing recommendations, e.g., payment recommendations, for health care service claims using a hierarchical auditing system in accordance with some embodiments of the inventive concept. Referring now to FIG. 3, information associated with a health care service, e.g., claim information including header information and line item information, is received at a customer data center 200 and provided, for example, to a primary auditing system sever 220 at block 300. According to some embodiments of the inventive concept, rules for evaluating line items in a claim may be distributed across a hierarchy of auditing systems including the primary auditing system server 220 and a secondary auditing system server 230 at block 305. While a single secondary auditing system is illustrated in FIG. 2, as explained above with respect to FIG. 1, multiple secondary auditing systems may be used in accordance with other embodiments of the inventive concept. The primary auditing system server 220 generates a first payment recommendation for one or more line items in a claim at block 310. At block 315 a determination is made whether to communicate the line item to the secondary auditing system server 240 by way of the remote connection system server 230. As described above, in some circumstances the remote connection system server 230 may not forward a claim to the secondary auditing system server 240 due to information contained in a claim level field that indicates that review by a secondary auditing system may not provide value, e.g., the payor's claims processing system is not configured to process recommendations from a secondary auditing system, a patient is self-insured, etc. In some embodiments, the line items in a claim may be tagged, which provides information to one or more secondary auditing systems. Specifically, a secondary auditing system may apply its rules to a line item informed by the information conveyed in the tag to evaluate the tagged line item for payment or use the information associated with the line item and tag in evaluating one or more other line items for payment in the same claim and/or other claims. If, for example, the primary auditing system server 220 makes a recommendation to pay in whole or in part, then the claim including the line item is communicated to the secondary auditing system server 240 with a tag indicating a recommendation has been made to pay, which may be interpreted by the rules running on the secondary auditing system server 240 as an instruction to obtain a second recommendation. If, however, the primary auditing system server 220 makes a recommendation to deny payment for the line item, then the claim including the line item may be communicated to the secondary auditing system server 240 with a tag indicating that a recommendation has been made to deny payment. This may be interpreted by the rules running on the secondary auditing system server 240 in various ways depending on the state of the tag assigned to the line. As described above, the secondary auditing system server 240 may use the line item for support in evaluating one or more lines in the same claim or other claim for payment based on the level of certainty with which the primary auditing system server 220 denied payment of the tagged line. Upon receiving a claim at the secondary auditing system server 240, a second payment recommendation is generated at block 320 using the secondary auditing system 240 for the one or more lines for which a first payment recommendation was generated at block 310. The remote connection server 230 merges the first payment recommendation with the second payment recommendation to generate a final payment recommendation at block 325.

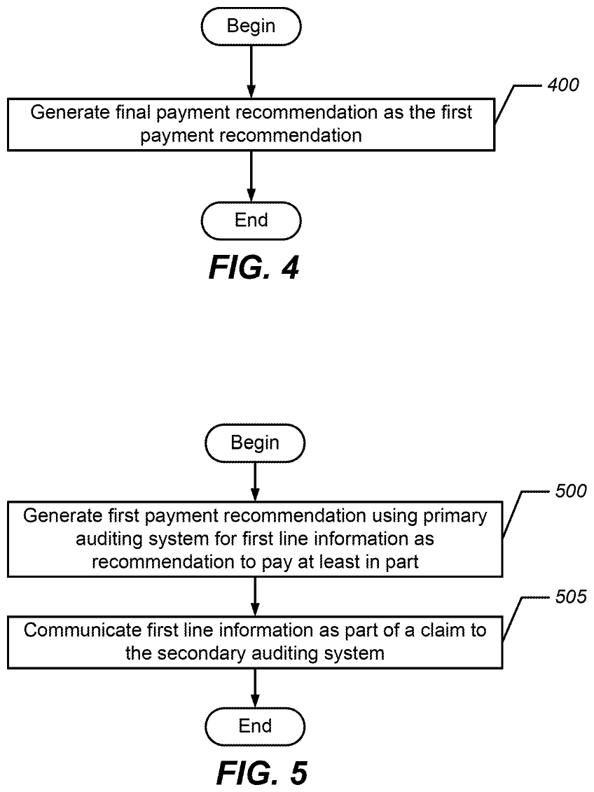

Referring to FIG. 4, the remote connection server 230 generates the final payment recommendation as the first payment recommendation generated by the primary auditing system server 220 when the first payment recommendation is a non-payment recommendation).

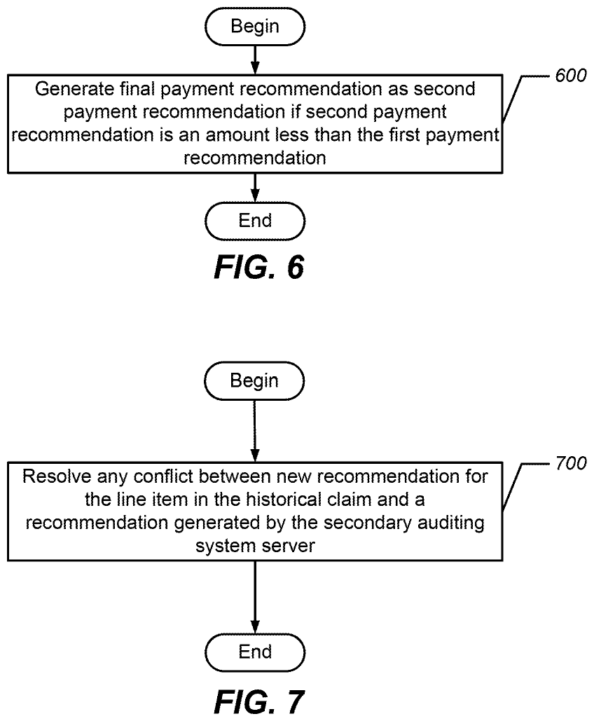

Referring now to FIG. 5, the primary auditing system server 220 may generate a recommendation to pay in whole or in part at block 500. When the primary auditing system server 220 generates a recommendation to pay a line item for a claim, then the line item may be communicated as part of a claim, for example, to the secondary auditing system server 240 at block 505 to obtain a second recommendation. Referring to FIG. 6, if the second payment recommendation generated by the second auditing system server 240 is less than an amount of the first recommendation, then the remote connection system server 235 may generate the final payment recommendation as the second payment recommendation at block 600, which may save the payor money when processing the claim for payment. Ultimately, whether the payor is able to use the reduced payment recommendation provided by the secondary auditing system server 240 may be dependent on licensing/contingency pricing models used by the payor.

As described above, the remote connection system server 230 may resolve any conflicts that may occur due to the primary auditing system having access to more historical claim data than may be available to the secondary auditing system. Thus, in general, if the secondary auditing system server 240 generates a recommendation to deny payment for a line item or pay less than an amount recommended by the primary auditing system server, then the remote connection system server 240 may accept the recommendation by the secondary auditing system server 240 as the final recommendation due to the secondary auditing system saving the payor money. But, referring now to FIG. 7, if the primary auditing system server 220 generates a new recommendation for a line item in a historical claim, then the remote connection system server 230 may resolve any conflict between the new recommendation for the line item in the historical claim and a recommendation generated by the secondary auditing system server 240 at block 700. As for non-historical claims, when the primary auditing system server 220 makes a non-pay recommendation, then that recommendation is taken as the final recommendation. When the primary auditing system server 220 and the secondary auditing system server 240 both make recommendations to pay in full or in part, then the remote connection server 230 may resolve any conflict based on the history data available to both the primary auditing system server 220 and the secondary auditing system server 240 along payment arrangements that have been defined, e.g., whether payment is licensed based or contingency based. As described above, the primary and secondary auditing system servers 220, 240 may have access to different history information depending on what information is available in databases and/or what information has been forwarded from the primary auditing system server 220 to the secondary auditing system server 240. The difference in available history information may favor, for example, a recommendation generated by the primary auditing system server 220 over a recommendation generated by the secondary auditing system server 240.

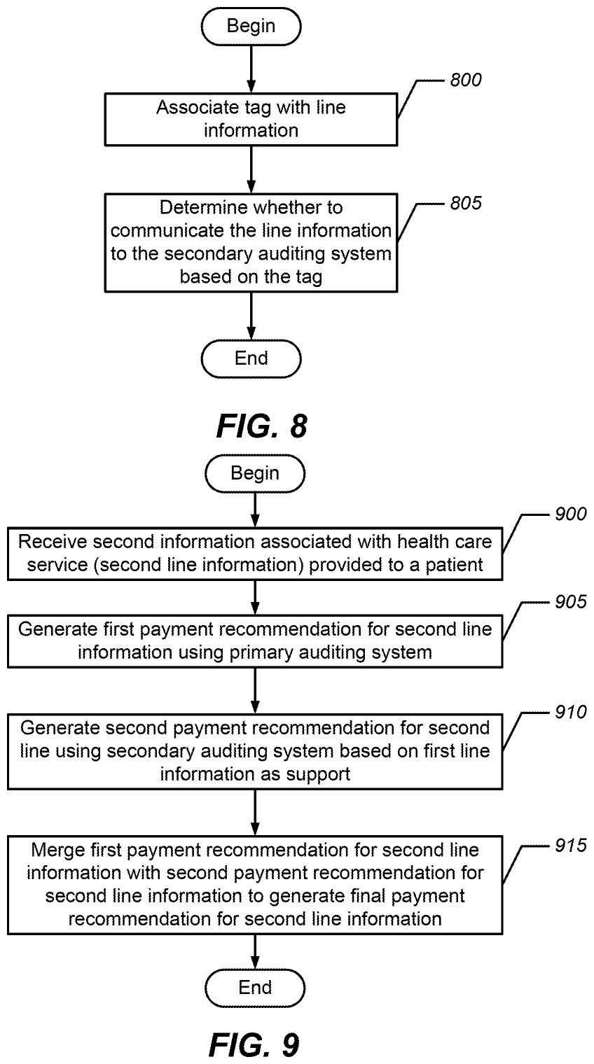

The remote connection system server 230 may further provide a tagging capability, which can be used to provide instructions that may be processed using the rules running on the secondary auditing system server 240. This may allow the secondary auditing module 245 and secondary auditing system server 240 to use information embedded or implicit in the tags to make payment recommendations on a particular tagged line or to use the tag and line information as support for making recommendations for other lines in the same claim and/or other claim. The tagging may be designed in a variety of ways. For example, any number of tags may be defined and each tag may take on any number of possible states. The greater the number of possible tags and the greater the number of possible states the greater the amount of information that can be conveyed to the secondary auditing module 245/secondary auditing system server 240 for interpretation by the rules running thereon. Moreover, as described above, in some embodiments, the remote connection server 230 may provide a filtering capability with respect to which claims are forwarded to the secondary auditing system server 240 based on claim level fields. In other embodiments, claims may be divided into lines and the tags associated with the individual lines may be used by the remote connection server 230 as a basis for forwarding individual lines to the secondary auditing system server 240 for evaluation. Such an approach may be more processor intensive as the remote connection server 230 would need to track which lines are sent to the secondary auditing system server 240 and reconstruct the original claim upon receiving recommendation results from the secondary auditing system server 240. But the information associated with the tags provides the remote connection system server 230 with a more complex and refined filtering capability with respect to what line items are forwarded to the secondary auditing system server 240. Referring now to FIG. 8, the remote connection system server 230 may associate a tag with a line item for a claim at block 500. The tag may be configured in one or more states to control the forwarding of the line item to the secondary auditing system 240. Thus, at block 805, the remote connection server 230 may determine whether to communicate a line item to the secondary auditing system based on a state of the tag.

For example, the primary auditing system server may recommend that payment be denied for a line item. If the denial is made with a high certainty, then it may still be useful to forward the line item to the secondary auditing system server 240 as the secondary auditing system server 240 may be able to use that line item information along with the denial recommendation as support in evaluating another line item in the same or another claim for payment. If, however, the denial is made with less certainty, then the line item may not be communicated to the secondary auditing system server 240 as it may not be useful or reliable for use as support information in evaluating other line items for payment. Thus, the use of tagging may allow for exceptions to the general rule of not forwarding line items for which the primary auditing system server 220 has made a non-payment recommendation to the secondary auditing system server 240 to obtain a second recommendation.

Referring to FIG. 9, the use of a line item as source information when evaluating another line item for payment will now be described. Operations begin at block 900 where a claim including a second line item is received at the primary auditing system server 220 and a first payment recommendation is generated using the primary auditing system server 220 at block 905. The claim including the second line item is forwarded to the secondary auditing system server 240 at block 910 and a second payment recommendation is generated for the second line item based on both the second line item information as well as information associated with a first line item that had a non-payment recommendation generated by the first auditing system server 220. But the non-payment recommendation was generated with sufficient certainty that the first line item was tagged with the tag configured in a state to allow the rules running on the secondary auditing system server to use the first line item as support in evaluating other line items. The remote connection system server 230 merges the first payment recommendation for the second line item and the second payment information for the second line item to generate a final payment recommendation for the second line item at block 915.

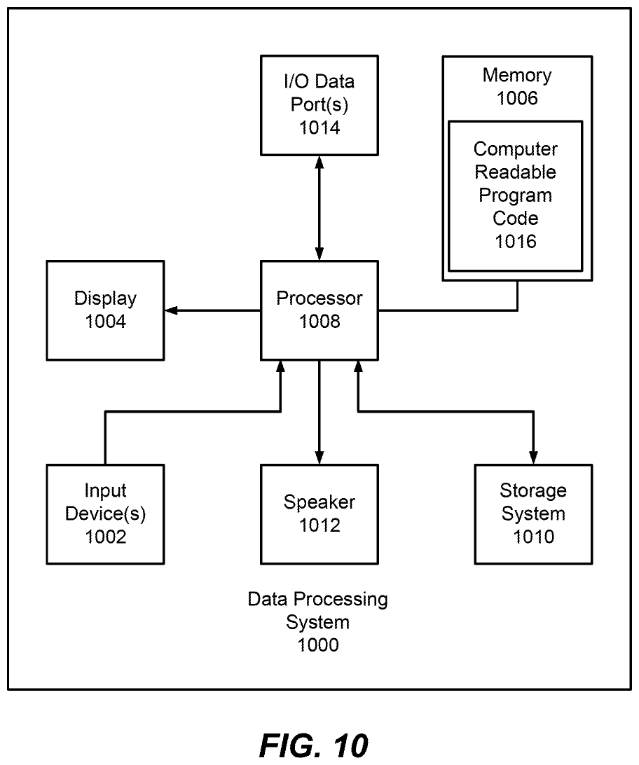

Referring now to FIG. 10, a data processing system 1000 that may be used to implement the primary auditing system server 220, the remote connection system server 235, and/or the secondary auditing system server 240 of FIG. 2, in accordance with some embodiments of the inventive concept, comprises input device(s) 1002, such as a keyboard or keypad, a display 1004, and a memory 1006 that communicate with a processor 1008. The data processing system 1000 may further include a storage system 1010, a speaker 1012, and an input/output (I/O) data port(s) 1014 that also communicate with the processor 1008. The processor 1008 may be, for example, a commercially available or custom microprocessor. The storage system 1010 may include removable and/or fixed media, such as floppy disks, ZIP drives, hard disks, or the like, as well as virtual storage, such as a RAMDISK. The I/O data port(s) 1014 may be used to transfer information between the data processing system 1000 and another computer system or a network (e.g., the Internet). These components may be conventional components, such as those used in many conventional computing devices, and their functionality, with respect to conventional operations, is generally known to those skilled in the art. The memory 1006 may be configured with computer readable program code 1016 to facilitate generating processing recommendations for health care service claims using a hierarchical auditing system according to some embodiments of the inventive concept.

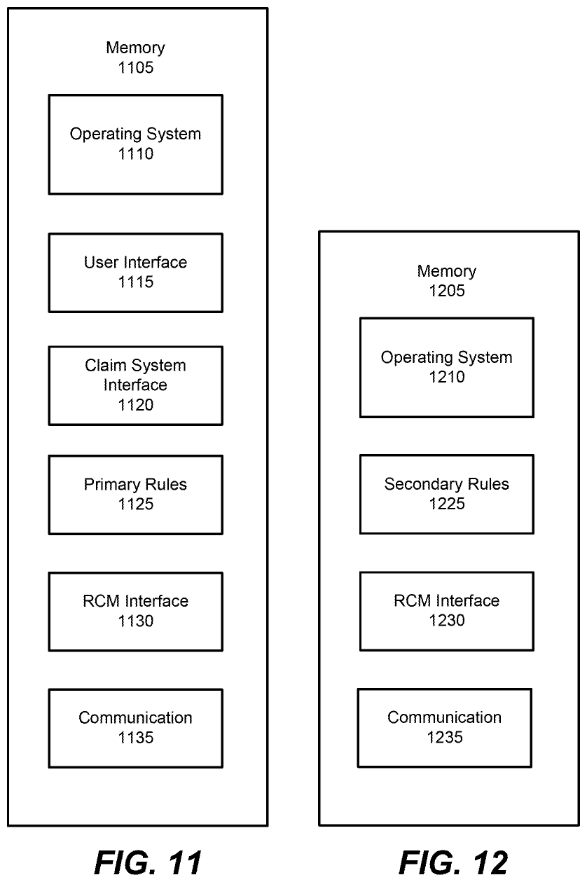

FIG. 11 illustrates a memory 1105 that may be used in embodiments of data processing systems, such as the primary auditing system server 220 and primary auditing module 225 of FIG. 2 and the data processing system 1000 of FIG. 10, respectively, to facilitate generating processing recommendations for health care service claims using a hierarchical auditing system according to some embodiments of the inventive concept. The memory 1105 is representative of the one or more memory devices containing the software and data used for facilitating operations of the primary auditing system server 220 described herein. The memory 1105 may include, but is not limited to, the following types of devices: cache, ROM, PROM, EPROM, EEPROM, flash, SRAM, and DRAM. As shown in FIG. 11, the memory 1105 may contain six or more categories of software and/or data: an operating system 1110, a user interface module 1115, a claim system interface module 1120, a primary rules module 1125, a remote connection module (RCM) interface 1130, and a communication module 1135. In particular, the operating system 1110 may manage the data processing system's software and/or hardware resources and may coordinate execution of programs by the processor. The user interface module 1115 may be configured to perform one or more of the operations described above with respect to the customer data center 100, customer data center 200, primary auditing system 105, primary auditing system server 220, primary auditing module 225, and the flowcharts of FIGS. 3-9 regarding presenting payment recommendations for claims or claim line items to a payor. The claim system interface module 1120 may be configured to perform one or more of the operations described above with respect to the claim systems 102a, 102b, and 102c, customer data center 100, claim systems 202a, 202b, and 202c, customer data center 200, primary auditing system 105, primary auditing system server 220, primary auditing module 225, and the flowcharts of FIGS. 3-9 regarding receiving claims for services performed by providers. The primary rules module 1125 may be configured to perform one or more of the operations described above with respect to the primary auditing system 105, primary auditing system server 220, primary auditing module 225, and the flowcharts of FIGS. 3-9 regarding generating a payment recommendation for a claim line item using rules in the primary auditing system. The RCM interface module 1130 may be configured to perform one or more of the operations described above with respect to the primary auditing system server 220, primary auditing module 225, remote connection system 110, the remote connection system server 230, the remote connection module 235, and the flowcharts of FIGS. 3-9 regarding interaction between the primary auditing system and the remote connection system. The communication module 1135 may be configured to perform one or more operations described above with respect to the primary auditing system 105, primary auditing system server 220, primary auditing module 225, and the flowcharts of FIGS. 3-9 regarding the ability of the primary auditing system to communicate with other entities including a customer data center and a remote connection system.

FIG. 12 illustrates a memory 1205 that may be used in embodiments of data processing systems, such as the secondary auditing system server 240 and secondary auditing module 245 of FIG. 2 and the data processing system 1000 of FIG. 10, respectively, to facilitate generating processing recommendations for health care service claims using a hierarchical auditing system according to some embodiments of the inventive concept. The memory 1205 is representative of the one or more memory devices containing the software and data used for facilitating operations of the secondary auditing system server 240 described herein. The memory 1205 may include, but is not limited to, the following types of devices: cache, ROM, PROM, EPROM, EEPROM, flash, SRAM, and DRAM. As shown in FIG. 12, the memory 1205 may contain four or more categories of software and/or data: an operating system 1210, a secondary rules module 1225, an RCM interface 1230, and a communication module 1235. In particular, the operating system 1210 may manage the data processing system's software and/or hardware resources and may coordinate execution of programs by the processor. The secondary rules module 1225 may be configured to perform one or more of the operations described above with respect to the second through Nth auditing system 115a . . . , 115n, secondary auditing system server 240, secondary auditing module 245, and the flowcharts of FIGS. 3-9 regarding generating a payment recommendation for a claim line item using rules in the secondary auditing system. The RCM interface module 1230 may be configured to perform one or more of the operations described above with respect to the secondary system server 240, secondary auditing module 245, remote connection system 110, the remote connection system server 230, the remote connection module 235, and the flowcharts of FIGS. 3-9 regarding interaction between the secondary auditing system and the remote connection system. The communication module 1135 may be configured to perform one or more operations described above with respect to the second through Nth auditing system 115a . . . , 115n, secondary auditing system server 240, secondary auditing module 245, and the flowcharts of FIGS. 3-9 regarding the ability of the secondary auditing system to communicate with other entities including a primary auditing system and a remote connection system.

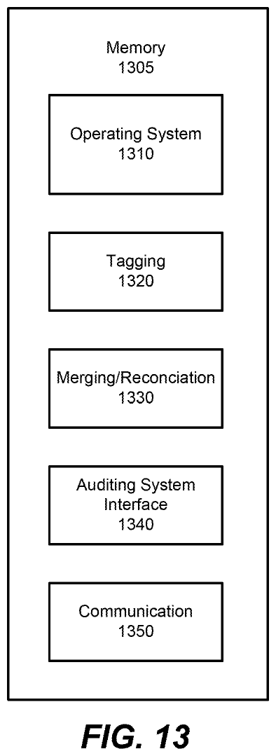

FIG. 13 illustrates a memory 1305 that may be used in embodiments of data processing systems, such as the remote connection system server 230 and remote connection module 235 of FIG. 2 and the data processing system 1000 of FIG. 10, respectively, to facilitate generating processing recommendations for health care service claims using a hierarchical auditing system according to some embodiments of the inventive concept. The memory 1305 is representative of the one or more memory devices containing the software and data used for facilitating operations of the remote connection system server 230 described herein. The memory 1305 may include, but is not limited to, the following types of devices: cache, ROM, PROM, EPROM, EEPROM, flash, SRAM, and DRAM. As shown in FIG. 13, the memory 1205 may contain five or more categories of software and/or data: an operating system 1310, a tagging module 1320, a merging/reconciliation module 1330, an auditing system interface module 1340, and a communication module 1350. In particular, the operating system 1310 may manage the data processing system's software and/or hardware resources and may coordinate execution of programs by the processor. The tagging module may be configured to perform one or more of the operations described above with respect to the remote connection system 110, the remote connection system server 230, the remote connection module 235, and the flowcharts of FIGS. 3-9 regarding the ability to associate a tag with a line item and to configure the tag in a state to provide information to a secondary auditing system. The merging/reconciliation module 1330 may be configured to perform one or more of the operations described above with respect to the remote connection system 110, the remote connection system server 230, the remote connection module 235, and the flowcharts of FIGS. 3-9 regarding generating a final payment recommendation based on payment recommendations provided by a primary auditing system and one or more secondary auditing systems. The auditing system interface module 1340 may be configured to perform one or more of the operations described above with respect to the remote connection system 110, the remote connection system server 230, the remote connection module 235, and the flowcharts of FIGS. 3-9 regarding interaction between the remote connection system and the primary and secondary auditing systems. The communication module 1350 may be configured to perform one or more operations described above with respect to the remote connection system server 230, the remote connection module 235, and the flowcharts of FIGS. 3-9 regarding the ability of the remote connection system to communicate with the primary auditing system and secondary auditing system or between two secondary auditing systems.

Although FIGS. 10-13 illustrate hardware/software architectures that may be used in data processing systems, such as the primary auditing system server 220 and primary auditing module 225 of FIG. 2, the secondary auditing system server 240 and secondary auditing module 245 of FIG. 2, the remote connection system server 230 and remote connection module 235 of FIG. 2, and the data processing system 1000 of FIG. 10, respectively, in accordance with some embodiments of the inventive concept, it will be understood that the present invention is not limited to such a configuration but is intended to encompass any configuration capable of carrying out operations described herein.

Computer program code for carrying out operations of data processing systems discussed above with respect to FIGS. 1-11 may be written in a high-level programming language, such as Python, Java, C, and/or C++, for development convenience. In addition, computer program code for carrying out operations of the present invention may also be written in other programming languages, such as, but not limited to, interpreted languages. Some modules or routines may be written in assembly language or even micro-code to enhance performance and/or memory usage. It will be further appreciated that the functionality of any or all of the program modules may also be implemented using discrete hardware components, one or more application specific integrated circuits (ASICs), or a programmed digital signal processor or microcontroller.

Moreover, the functionality of the primary auditing system server 220 of FIG. 2, the secondary auditing system server 240, the remote connection system server 230 of FIG. 2, and the data processing system 1000 of FIG. 10 may each be implemented as a single processor system, a multi-processor system, a multi-core processor system, or even a network of stand-alone computer systems, in accordance with various embodiments of the inventive concept. Each of these processor/computer systems may be referred to as a “processor” or “data processing system.”

The data processing apparatus described herein with respect to FIGS. 1-13 may be used to facilitate generating processing recommendations for health care service claims using a hierarchical auditing system according to some embodiments of the inventive concept described herein. These apparatus may be embodied as one or more enterprise, application, personal, pervasive and/or embedded computer systems and/or apparatus that are operable to receive, transmit, process and store data using any suitable combination of software, firmware and/or hardware and that may be standalone or interconnected by any public and/or private, real and/or virtual, wired and/or wireless network including all or a portion of the global communication network known as the Internet, and may include various types of tangible, non-transitory computer readable media. In particular, the memory 1105 when coupled to a processor includes computer readable program code that, when executed by the processor, causes the processor to perform operations including one or more of the operations described herein with respect to FIGS. 1-9.

Some embodiments of the inventive concept may provide a hierarchical auditing system that may provide a payor responsible for making payment for claims for services or goods, such as health care insurance claims, the ability to enhance their primary auditing system, which provides rules for making payment recommendations for line items in the claims. Specifically, the primary auditing system may be supplemented with one or more secondary auditing systems that provide additional rules for evaluating claim line items for payment. These secondary auditing systems may be accessed through the cloud for convenient and expeditious access to newly developed rules. The payor may be offered access to the rules on a contingency basis where the payor is charged a percentage of the savings obtained through application of the rules in the secondary auditing system.

Further Definitions and Embodiments:

In the above-description of various embodiments of the present inventive concept, it is to be understood that the terminology used herein is for the purpose of describing particular embodiments only and is not intended to be limiting of the invention. Unless otherwise defined, all terms (including technical and scientific terms) used herein have the same meaning as commonly understood by one of ordinary skill in the art to which this inventive concept belongs. It will be further understood that terms, such as those defined in commonly used dictionaries, should be interpreted as having a meaning that is consistent with their meaning in the context of this specification and the relevant art and will not be interpreted in an idealized or overly formal sense expressly so defined herein.

The flowchart and block diagrams in the figures illustrate the architecture, functionality, and operation of possible implementations of systems, methods, and computer program products according to various aspects of the present inventive concept. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The terminology used herein is for the purpose of describing particular aspects only and is not intended to be limiting of the inventive concept. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Like reference numbers signify like elements throughout the description of the figures.

In the above-description of various embodiments of the present inventive concept, aspects of the present inventive concept may be illustrated and described herein in any of a number of patentable classes or contexts including any new and useful process, machine, manufacture, or composition of matter, or any new and useful improvement thereof. Accordingly, aspects of the present inventive concept may be implemented entirely hardware, entirely software (including firmware, resident software, micro-code, etc.) or combining software and hardware implementation that may all generally be referred to herein as a “circuit,” “module,” “component,” or “system.” Furthermore, aspects of the present inventive concept may take the form of a computer program product comprising one or more computer readable media having computer readable program code embodied thereon.

Any combination of one or more computer readable media may be used. The computer readable media may be a computer readable signal medium or a computer readable storage medium. A computer readable storage medium may be, for example, but not limited to, an electronic, magnetic, optical, electromagnetic, or semiconductor system, apparatus, or device, or any suitable combination of the foregoing. More specific examples (a non-exhaustive list) of the computer readable storage medium would include the following: a portable computer diskette, a hard disk, a random access memory (RAM), a read-only memory (ROM), an erasable programmable read-only memory (EPROM or Flash memory), an appropriate optical fiber with a repeater, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

The description of the present inventive concept has been presented for purposes of illustration and description, but is not intended to be exhaustive or limited to the inventive concept in the form disclosed. Many modifications and variations will be apparent to those of ordinary skill in the art without departing from the scope and spirit of the inventive concept. The aspects of the inventive concept herein were chosen and described to best explain the principles of the inventive concept and the practical application, and to enable others of ordinary skill in the art to understand the inventive concept with various modifications as are suited to the particular use contemplated.

What is claimed is:

1. A method for operating a computer-implemented claims processing system, comprising:
providing a primary computer-implemented auditing system having a first set of rules for determining responsibility for payment for health care services;
providing a secondary computer-implemented auditing system having a second set of rules for determining responsibility for payment for health care services, the second set of rules not being identical to the first set of rules;
receiving, at the primary computer-implemented auditing system, information associated with a claim for a health care service provided to a patient;
generating, using primary computer-implemented auditing system, a first payment recommendation for the health care service provided to the patient based on the first set of rules and the information associated with the health care service provided to the patient;
determining that sending the information regarding the health care service to the secondary computer-implemented auditing system will not provide value in determining responsibility for the payment for the health care service provided to the patient; and
generating a final payment recommendation for the health care service based only on the first payment recommendation;
wherein performance of the computer-implemented claims processing system is improved by not requiring the primary computer-implemented auditing system to store the second set of rules, nor requiring the secondary computer-implemented auditing system to provide a second payment recommendation.

2. The method of claim 1, wherein:
the claim for a health care service has at least one line item;
generating, using the primary computer-implemented auditing system, a first payment recommendation for the at least one line item of the health care service based on the first set of rules and the information associated with the health care service;
determining that sending the information regarding the at least one line item of the health care service to the secondary computer-implemented auditing system will not provide value in determining responsibility for the payment for the health care service; and
generating a final payment recommendation for the at least one line item of the health care service based only on the first payment recommendation.

3. The method of claim 1, and further comprising:
subsequent to determining that sending the information regarding the health care service to the secondary computer-implemented auditing system will not provide value, forwarding the information regarding the health care service to the secondary computer-implemented auditing system wherein the secondary computer-implemented auditing system uses the information regarding the health care service to evaluate a claim for payment for a different health care service provided to the patient.

4. The method of claim 1, wherein determining that sending the information regarding the health care service to the secondary computer-implemented auditing system will not provide value comprises determining that the claims processing system is not configured to accept a recommendation from the secondary computer-implemented auditing system.

5. The method of claim 1, wherein determining that sending the information regarding the health care service to the secondary computer-implemented auditing system will not provide value comprises determining that the patient is self-insured.

6. The method of claim 1, and further comprising:
the claim for a health care service has at least one line item;
subsequent to determining that sending the information regarding the health care service to the secondary computer-implemented auditing system will not provide value, forwarding the information regarding the health care service to the secondary computer-implemented auditing system wherein the secondary computer-implemented auditing system uses the information regarding the health care service to evaluate a claim for payment for a different line item of the health care service provided to the patient.

7. The method of claim 1, and further comprising:
the claim for a health care service has at least one line item;
generating, using the primary computer-implemented auditing system, a first payment recommendation for a different line item of the health care service based on the first set of rules and the information associated with the health care service;
subsequent to the determining, forwarding the information regarding at least the different line item of the health care service to the secondary computer-implemented auditing system;
generating, using the secondary computer-implemented auditing system, a second payment recommendation for the different line item based on the second set of rules and the information associated with the health care service; and
merging the first payment recommendation for the different line item with the second payment recommendation for the different line item to generate a final payment recommendation for the different line item of the health care service.

8. A method for operating a computer-implemented claims processing system, comprising:
providing a primary computer-implemented auditing system having a first set of rules for determining responsibility for payment for health care services;
providing a secondary computer-implemented auditing system having a second set of rules for determining responsibility for payment for health care services, the second set of rules not being identical to the first set of rules;
receiving at the primary computer-implemented auditing system, information associated with a health care service provided to a patient;
generating, using the primary computer-implemented auditing system, a first payment recommendation for the health care service based on the first set of rules and the information associated with the health care service;
communicating the information associated with the health care service to the secondary computer-implemented auditing system via a remote connection system connecting the primary computer-implemented auditing system and the secondary computer-implemented auditing system;
generating, using the secondary computer-implemented auditing system, a second payment recommendation for the health care service based on the second set of rules and the information associated with the health care service; and
merging the first payment recommendation with the second payment recommendation to generate a final payment recommendation for the health care service;
wherein performance of the computer-implemented claims processing system is improved by not requiring the primary computer-implemented auditing system to store the second set of rules.

9. The method of claim 8, wherein the first payment recommendation is a recommendation not to pay for the health care service; and
wherein merging the first payment recommendation with the second payment recommendation to generate the final payment recommendation for the health care service comprises:
generating the final payment recommendation based on the first payment recommendation without modification by the second payment recommendation.

10. The method of claim 8, further comprising:
associating a tag with the information associated with the health care service, the tag providing instructions for processing the information associated with the health care service in the secondary computer-implemented auditing system.

11. The method of claim 10, wherein the information is first information, the health care service is a first health care service, and the tag is a first tag, the method further comprising:
receiving second information associated with a second health care service provided to the patient;
associating a second tag with the second information associated with the second health care service, the second tag providing instructions for processing the second information associated with the second health care service in the secondary computer-implemented auditing system;
generating, using the primary computer-implemented auditing system, a first payment recommendation for the second health care service based on the first set of rules and the second information associated with the second health care service;
generating, using the secondary computer-implemented auditing system, a second payment recommendation for the second health care service based on the second set of rules, the second information associated with the second health care service, the first tag, the second tag, and the first information associated with the first health care service; and
merging the first payment recommendation for the second health care service with the second payment recommendation for the second health care service to generate a final payment recommendation for the second health care service.

12. The method of claim 8, wherein the information associated with the health care service corresponds to a line item in a health care claim for payment; and
wherein communicating the information associated with the health care service to the secondary computer-implemented auditing system comprises communicating the health care claim for payment to the secondary computer-implemented auditing system.

13. The method of claim 8, wherein the information associated with the health care service corresponds to a line item in a health care claim for payment; and
wherein communicating the information associated with the health care service to the secondary computer-implemented auditing system comprises communicating the line item to the secondary computer-implemented auditing system without communicating other line items of the health care claim for payment to the secondary computer-implemented auditing system.

14. The method of claim 8, wherein the first payment recommendation is a recommendation to pay for the health care service in whole or in part;
wherein the second payment recommendation is not to pay in whole or in part for the health care service; and
wherein merging the first payment recommendation with the second payment recommendation to generate the final payment recommendation for the health care service comprises:
generating the final payment recommendation based on the second payment recommendation when the second payment recommendation is an amount less than the first payment recommendation.

15. The method of claim 8, wherein the first payment recommendation is a recommendation to pay for the health care service in whole or in part based on the first set of rules, the information associated with the health care service, and historical information associated with the health care service, which is unavailable to the secondary computer-implemented auditing system;
wherein the second payment recommendation is not to pay in whole or in part for the health care service; and
wherein merging the first payment recommendation with the second payment recommendation to generate the final payment recommendation for the health care service comprises:
generating the final payment recommendation based on the first payment recommendation without modification by the second payment recommendation.

16. A computer-implemented system, comprising:
a primary computer-implemented auditing system comprising:
a first processor; and
a first memory coupled to the first processor and comprising first computer readable program code embodied in the first memory that is executable by the first processor to perform operations comprising:
store a first set of rules to determine responsibility for payment for health care services;
receive information associated with a health care service provided to a patient;
generate a first payment recommendation for the health care service based on the first set of rules and the information associated with the health care service; and
communicate the information associated with the health care service to a secondary computer-implemented auditing system via a remote connection system;
the second computer-implemented auditing system comprising:
a second processor; and
a second memory coupled to the second processor and comprising computer readable program code embodied in the second memory that is executable by the second processor to perform operations comprising:
store a second set of rules to determining responsibility for payment for health care services, the second set of rules not being identical to the first set of rules;
receive information associated with a health care service provided to a patient from the computer-implemented auditing system via the remote connection system;
generate a second payment recommendation for the health care service based on the second set of rules and the information associated with the health care service; and
send the second payment recommendation to the primary computer-implemented auditing system via the remote connection system;
the remote connection system connecting the primary computer-implemented auditing system and the secondary computer-implemented auditing system;
wherein the first computer readable program code is further executable by the first processor to receive the second payment recommendation from the secondary computer-implemented auditing system, and to merge the first payment recommendation with the second payment recommendation to generate a final payment recommendation for the health care service.

17. The computer-implemented system of claim 16, wherein the first payment recommendation is a recommendation not to pay for the health care service; and
wherein to merge the first payment recommendation with the second payment recommendation to generate the final payment recommendation for the health care service comprises:
to generate the final payment recommendation as the first payment recommendation without modification by the second payment recommendation.

18. The computer-implemented system of claim 16,
wherein the first computer readable program code is further executable by the first processor to associate a tag with the information associated with the health care service, the tag providing instructions for processing the information associated with the health care service in the secondary computer-implemented auditing system; and
wherein the second computer readable program code is further executable by the second processor to generate the second payment recommendation based on the tag.

19. The computer-implemented system of claim 16, wherein the information is first information, the health care service is a first health care service, and the tag is a first tag, and
wherein the first computer readable program code is further executable by the first processor to:
receive second information associated with a second health care service provided to the patient;
associate a second tag with the second information associated with the second health care service, the second tag providing instructions for processing the second information associated with the second health care service in the secondary computer-implemented auditing system;
generate a first payment recommendation for the second health care service based on the first set of rules and the second information associated with the second health care service;
wherein the second computer readable program code is further executable by the second processor to generate the second payment recommendation for the second health care service based on the second set of rules, the second information associated with the second health care service, the first tag, the second tag, and the first information associated with the first health care service; and
wherein the first computer readable program code is further executable by the first processor to merge the first payment recommendation for the second health care service with the second payment recommendation for the second health care service to generate a final payment recommendation for the second health care service.

20. The computer-implemented system of claim 16, wherein the information associated with the health care service corresponds to a line item in a health care claim for payment; and
wherein the first computer readable program code is further executable by the first processor to communicate the line item in the health care claim to the secondary computer-implemented auditing system.
